(19) United States
(12) Patent Application Publication
(10) Pub. No.: US 2012/0258526 A1
(43) Pub. Date: Oct. 11, 2012

(54) PACKING MATERIAL COMPRISING STARCH-MODIFIED POLYURETHANE FOR THE BIOFILTRATION OF ORGANIC COMPOUNDS PRESENT IN GASEOUS OR LIQUID EFFLUENTS, PRODUCTION METHODS THEREOF AND BIOFILTRATION SYSTEM

(76) Inventors: Olga Brígida Gutierrez Acosta, Irapuato (MX); Vladimir Alonso Escobar Barrios, San Luis Potosi (MX); Sonia Lorena Arriaga Garcia, San Luis Potosi (MX)

(21) Appl. No.: 13/515,450

(22) PCT Filed: Dec. 16, 2010
(86) PCT No.: PCT/MX2010/000155

§ 371 Date: Jun. 12, 2012

(30) Foreign Application Priority Data
Dec. 17, 2009 (MX) MX/A/2009/013966

(51) Int. Cl. B01J 20/26; B01J 20/30; B29C 67/20; C12M 1/00

(52) U.S. Cl. 435/289.1; 252/184; 264/321

(57) ABSTRACT

The invention relates to a packing material for biofilters, having a polyurethane polymer and starch. The packing material is resistant to compaction, can sorb pollutant organic compounds and reduces the start-up time of the biofilter. The packing material can be used as a substrate in the biofiltration of volatile and/or semi-volatile organic compounds present in gaseous or liquid effluents.

### CROSS REFERENCE TO RELATED APPLICATION

This application is a national stage entry of PCT/MX2010/000155 filed Dec. 16, 2010, under the International Convention claiming priority over Mexican Patent Application No. MX/a/2009/013966 filed Dec. 17, 2009.

### FIELD OF THE INVENTION

This invention relates to a packing material, based on starch-modified polyurethanes, which is inoculated with microorganisms capable of degrading organic compounds, individually or in mixture. The inoculated packaging material exhibits sorption capacity of organic compounds and is foamed sufficiently to promote contact between the contaminants and microorganisms.

### BACKGROUND OF THE INVENTION

The emissions of air pollutants and the discharge of them in water have serious impacts on human health and the environment. It was found that inhalation or ingestion of contaminants, even in low amounts, can cause irreversible damage to human health. Among the main health effects are asthma attacks, bronchitis, cancer, birth defects and heart diseases.

Volatile organic compounds and semi-volatile organic compounds (VOCs) are easy to find in the atmosphere as vapor and gaseous or liquid phases, and usually have a high toxicity. VOCs are the second most diverse and abundant class of atmospheric emissions, after the suspended particles, moreover, due to their physicochemical properties can be dissolved in water contributing to its pollution. Among these compounds are aromatics, alkanes, alkenes, chlorinated compounds, alcohols, aldehydes and ketones, esters, among others. In addition, these compounds are so varied that its effects are not fully known. For example, it is known that benzene has carcinogenic properties.

Biological treatment of such pollutants present in gaseous or liquid effluents is a promising alternative due to its low operating costs and reduced environmental impact. Added to this is an alternative treatment of gaseous and liquid effluents contaminated with which to achieve high removal efficiencies or transforming VOC biodegradation by aerobic pathway to less harmful products such as water, carbon dioxide and biomass (Chan, W., Lu, M., J. Polym. Environm., 2005, 13 (1), 7-17 and Desshusses M A, G. Hammer, Bioproc. Eng 1994, 9, 141-146). Microorganisms which catalyze the oxidation of VOCs can be supported on the packing material forming a biofilm.

In general, we can say that both, the type of organism as the type of packaging used in biofilters for VOC treatment, are the two main parties contributing to the reactor's performance. Elimination capacities (EC), obtained in a biofilter, are a function of the physicochemical characteristics of the packing material. It has been proposed the use of different types of packing material, which may be natural or synthetic. It is noteworthy that in the case of mineral materials is required to add nutrients for maintaining the activity of the microorganisms, which limits their application on industrial scale because operating costs are increased. Furthermore, the packing material should have some water retention capacity, porosity and physicochemical resistance over a wide pH range.

Various studies or systems have been described using inoculated packing material for the degradation of some contaminants.

By way of example, Canadian Patent No. 2,211,564 discloses a filter medium comprising a mixture of a) pelletized peat, b) a binder such as poly (vinyl alcohol), poly (acrylic acid), polyacrylamide, silicates, cement or guar gum, c) buffering agent, d) moisture-retaining material and e) a particular material that adsorbs not degraded contaminants. This media was used for supporting microorganisms. However, the use of many elements makes this invention has a high manufacturing cost, so the cost benefit does not meet the expectations about the system. It should be noted that this document does not suggest or disclose the use of starch-modified polyurethanes.

Furthermore, Sakuma et al. (Sakuma, T., Hattori, T. and Deshusses, MJ Air & Waste Manage. Assoc, 2006, 56, 1567-1575) studied and compared different packaging materials for the biofiltration of pollutants in air. Ceramics, minerals and polyurethane foams were compared during the biodegradation of toluene. It was concluded that the polyurethane foam had the lowest performance in the removal of VOCs compared to other materials. Said polyurethane foam was not modified and had a reduced water retention capacity, coupled it did not provide carbon or an energy source for the microorganisms.

An essential feature in a packaging material for biofilters is their capacity to retain moisture, which is essential to promote microbial activity during biodegradation. In the prior art has proposed several alternatives to achieve these characteristics.

Firstly, the international application WO 02/089959 describes the process and the system for the biofiltration of volatile organic compounds. The described biofilter material is based on fish shells which has a high content of calcareous material capable of retaining moisture. Although there is some level of moisture retention, such material does not provide an energy source for sustaining the microorganisms involved in biodegradation. It should be noted that the document did not use a material such as starch-modified polyurethanes.

Furthermore, the international application WO 02/085499 describes a biofiltration system for the removal of VOCs using a porous material based on polymeric materials. Such polymeric materials were selected from the group of polyethers, polyesters, polyethylene or polypropylene. Despite the foregoing, this system does not provide energy sources for the survival of the microorganisms and also does not describe the use of modified polyurethane.

Related to the use of polyurethane as packing material for biofilters, several alternatives based on polyurethane compounds have been used due to their chemical and mechanical characteristics. Conventionally, polyurethanes have been synthesized by reacting polyols with polyisocyanate in stoichiometric proportions or unequal NCO:OH ratios, in order to leave the isocyanate group (NCO) available, which can be used for any subsequent reaction. These materials are known as polyurethane prepolymers.

The polyurethane prepolymers are also known as polyisocyanate prepolymers, and are widely used in applications where elastomeric properties are required. The elastomeric characteristics relate to the material's ability to withstand mechanical stresses regaining his form after they have been freed from the effort.

The elastomeric behavior is important when the material will be subject to such efforts to bear and dissipate without any detrimental effects on material structure. In this sense, the polyurethane foams are excellent materials to withstand the mechanical stresses.

Another key feature of the packaging materials used in biofilters is to be materials capable of withstanding mechanical degradation in order to prevent clogging of the biofilter, which reduces performance. In this regard, various materials have been proposed both natural and synthetic.

Natural materials such as peat, wood, compost and soil have been used as a support of microorganisms. However, although these media provide nutrients to support microbial activity, the consumption of these over time can result in a decrease in mechanical strength, resulting in compaction of the bed, mass transfer problems, and clogging of the biofilter. This results in the presence of large pressure drops that bring high energy requirements for passing gas or liquid streams of volatile or semi-volatile compounds through the bed, in this case operating costs increase.

Mineral materials such as perlite, activated carbon, vermiculite and others, have also been used as packing materials in biofilters, taking advantage over natural materials by providing mechanical strength, but they do not provide nutrients that contribute to the growth of microorganisms or biomass increase, therefore it is required to add nutrients for the biofiltration process.

By way of example, British Patent GB 1243352 describes a filter for removing gases and vapors from the air, and for deodorizing the air, which comprises a porous material with a redox catalytic system. Among the materials used are optionally polyurethane and starch, which are not associated with each other, neither provide an active site for the biodegradation of organic compounds. The degradation is carried out through chemical interactions (redox), considering therefore a physical-chemical treatment of emissions.

Furthermore, US patent application 2003140794 A1, discloses a sponged filter for treat impurities, toxins and odors from air and water, wherein the main component is polyvinyl alcohol, which is processed and mixed with starch. However, this mixture is not sufficient to carry out filtering and requires adding activated carbon and formaldehyde to complete the paste, which must be heated and acetylated with polyurethane. Said patent application does not represent a prior art close to the present invention since it is not a packaging material used for the biotreatment of contaminants, simply, this packing is used for carrying out sorption processes in air and water pollutants. It also requires additional elements, and the base material is polyvinyl alcohol

Another example of biofilters materials is disclosed in Korean KR 100433644 (B1) and Korean Patent Application KR 200341511, wherein methods for preparing a porous polymer for a biofilter for the removal of contaminants are proposed. In said methods a small amount of polymer is used, together with aldehyde-based compounds, activated carbon or zeolites and is mixed with dextrin or starch, adding a foaming agent and acid to said mixture and finally the starch or dextrin is removed. However, the manufacturing process the material is in three steps, which increases costs and presents difficulty in its preparation. Moreover, during the synthesis of that material the organic compound is removed, which in the present invention is starch and is an essential element in the invention, since it is a carbon source for the microorganisms involved in biodegradation of organic compounds. Therefore, these Korean documents do not affect the novelty or inventive step of this application.

As seen from the documents previously mentioned, synthetic materials used are polyethylene, polypropylene and polyurethane. Nevertheless, no document exists in that describes or suggests that the polyurethane has been modified starch in order to improve the water retention capacity and sorption of organic contaminants, and that also serve as carbon source and energy for the supported microorganisms. Thus, the present invention overcomes and satisfies the need of a biofilter packing material at low cost and easy preparation, where the essential constituents are starch-modified polyurethane, wherein said packaging material can be inoculated with microorganisms capable of degrading volatile organic compounds and semi-volatile, single or mixed, which may be present in gaseous or liquid contaminated streams. The inoculated packaging material exhibits sorption capacity of organic compounds and is sufficiently foamed to promote contact between the pollutants and microorganisms.

The material can be used in different bioreactors configurations involving a microorganism carrier material, for example, slurry bed, two-phase partition liquid-solid, fluidized bed, among others. In addition, such bioreactors can be operated in batch and/or continuous mode. Said material has characteristics of shows minimum compaction, adequate water retention capacity and prevents high backpressure; in addition, the starch present in the material serves as a carbon and energy source for the microorganisms. Therefore, is capable of maintaining microbial activity for carrying out the process of biofiltration of volatile and semi-volatile organic compounds present in polluted gaseous or liquid effluents effectively and with short starting time. Finally, we mention that part of the data of the present application were presented at the Congress Biotechniques for Air Pollution Control, Sep. 28, 2009, in the work entitled “Effect of polyamine on adsorption and degradation of toluene by biocomposite based on natural fiber” and “Biodegradation of a mixture of hydrocarbon vapors using a modified polymeric support”, where no mention was made regarding the starch or reveal the key aspects of the invention.

### FIGURES DESCRIPTION

FIG. 1. This figure shows the biodegradation rate of hexane, toluene and methyl ethyl ketone (MEK) with the material of the formulation G. The abscissa axis shows the time in days, and the y-axis shows the dimensionless concentration C/Co.

Symbols: —▴—hexane, 0%; —▪—toluene, 0%; ——MEK, 0%; —Δ—hexane control, 0%; —□—toluene control 0%; —∘—MEK control, 0%.

FIG. 2. This figure shows the biodegradation rate for hexane, toluene and methyl ethyl ketone (MEK) with the packing material according to formulation B. The abscissa axis corresponds to time in days and the ordinate axis corresponds to the dimensionless concentration C/Co.

Symbols: —▴—hexane, 20%; —▪—toluene, 20%; ——MEK, 20%; —Δ—hexane control, 20%; —□—toluene control, 20%; —∘—MEK control, 20%.

FIG. 3. This figure shows the biodegradation rate for hexane, toluene and methyl ethyl ketone (MEK) with the packing material according to formulation C. The abscissa axis corresponds to time in days and the ordinate axis corresponds to the dimensionless concentration C/Co.

Symbols: —▴—hexane, 30%; —▪—toluene, 30%; ——MEK, 30%; —Δ—hexane control, 30%; —□—toluene control, 30%; —∘—MEK control, 30%.

FIG. 4. This figure shows the biodegradation rate for hexane, toluene and methyl ethyl ketone (MEK) with the packing material according to formulation D. The abscissa axis corresponds to time in days and the ordinate axis corresponds to the dimensionless concentration C/Co.

Symbols: —▴—hexane, 40%; —▪—toluene, 40%; ——MEK, 40%; —Δ—hexane control, 40%; —□—toluene control, 40%; —∘—MEK control, 40%.

FIG. 5. Microphotographs obtained by Scanning Electronic Microscopy (SEM) for formulations G, B, C and D at the end of the organic volatile compounds biodegradation experiments.

FIG. 6. This figure shows the effect of the contaminant feed load on the Elimination Capacity (EC). The abscissa axis corresponds to contaminant feed load (g/m⁻³/h) and the ordinate axis corresponds to the total EC (g/m⁻³/h) and the secondary ordinate axis corresponds to Elimination percentage, %.

FIG. 7. Microphotographs obtained by Scanning Electronic Microscopy (SEM) for the starch-modified polyurethane (formulation D), without microorganism at 300 μm (A), and with microorganism at day 60 of running the biofilter, at 50 μm, 300 μm and 1 mm (B, C and D, respectively).

### SUMMARY OF THE INVENTION

The present invention describes and claims a packaging material, which comprises a polyurethane and starch polymer, a foaming agent and water, wherein said packaging material comprises between 20% to 95% by weight of polyurethane, wherein said polyurethane polymer is obtained from a polyurethane prepolymer containing 10 to 18% of free isocyanate. Said polyurethane prepolymer is selected from the group consisting of products based on polyurethane prepolymers based on polyester or polyether polyurethane prepolymer from polyesters and polyurethane aqueous dispersions from polyols and/or polyesters. Additionally, the packing material of the present application comprises between 5% to 80% by weight of starch, wherein said starch is derived from any plant source such as but not limited to corn, oats, potatoes or rice. Likewise, said packaging material comprises from 0.5% to 2.0% by weight of foaming agent, wherein said foaming agent is an amine compound, preferably amine oxide, which is selected from the group consisting of coconut dimethyl amine oxide, dimethyl lauryl amine oxide, decyl dimethyl amine oxide, alkyl dimethyl amine oxide. In addition, said packaging material comprises between 0.25% to 1.0% by weight of water. An important feature is that the packaging material has a water retention capacity of between 12% to 61% by weight, based on the dry weight of packing material.

In a further embodiment of the invention, it is described and claimed a method for preparing the packing material described above, comprising the steps of: a) mixing a polyurethane prepolymer, starch, a foaming agent and water, b) enable foam and c) drying. Note that the ingredients used in this method are those described for the packing material as described above. Also, the mixing step can be, but is not limited to a mechanical mixing process at a temperature between 10° C. and 50° C. Furthermore, the foaming step can be carried out at a temperature between 10° C. and 50° C. and the drying step can be carried out at a temperature between 30° C. and 70° C. This method for preparing the packaging material comprises an additional step of shaping said packaging material wherein said packaging material form can be adapted by means of pressure or cutting.

It is a further object of the present invention to describe and claim a biofiltration system, which comprises a container containing a packing material as mentioned above, a culture medium and microorganisms capable of degrading volatile and semi-volatile organic compounds present in gaseous or liquid effluents. This container has inlet and outlet ports to admit and exhaust organic compounds. It is worth noting that the culture medium can be, but is not limited to a mineral medium. Furthermore, the microorganisms are yeasts, bacteria or fungi, or mixtures thereof. Additionally, this system is fed with volatile and/or semi-volatile organic compounds, being alone or in mixture, from 1 g m⁻³ h⁻¹ to 640 g m⁻³ h⁻¹. It should be noted finally that the biofiltration system has an efficiency of biodegradation of volatile and/or semi-volatile organic compounds of at least 85 percent compared to the initial concentration of these organic compounds.

### DETAILED DESCRIPTION OF THE INVENTION

The present invention provides a packaging material based on polyurethane modified with starch, which can be inoculated with microorganisms in order to biodegrade volatile and/or semi-volatile organic compounds present in gaseous and liquid effluents, such as but not limited to: methyl ethyl ketone, hexane, toluene, and many others. This modified polyurethane is very useful as a packing material because it can support and promote the growth of microorganisms because it contains an easily assimilable carbon and energy source. Therefore, the packaging material inoculated with microorganisms is used for the biodegradation of volatile and semi-volatiles organic compounds present in gaseous and liquid effluents, with high removal efficiency and short starting time.

In one embodiment of the present invention, the air stream contaminated with volatile and/or semi-volatile organic compounds can be fed to the bioreactor, and be passed through the packing material described in this application and which has previously been inoculated with a microorganisms. Subsequently, the bio-treated air stream leaves the biofilter. The biodegradation of volatile organic compounds and/or semi-volatile can be assessed, for example by gas chromatography, taking samples from the input and output ports of the bioreactor.

In a further embodiment of the present invention, the liquid stream contaminated with volatile and/or semi-volatile organic compounds can be fed to the bioreactor, and be passed through the packing material described in this application, which has been previously inoculated with microorganisms. Subsequently, the bio-treated liquid stream leaves the biofilter. Biodegradation of organic compounds is evaluated by gas chromatography or high resolution liquid chromatography, taking samples in the input and output ports of the bioreactor.

The microorganisms used in the packing material are all those capable of using starch as carbon and energy source, with the added ability of biodegrade pollutants based on organic compounds from liquid or gaseous effluents.

Thus, a first aspect of the present invention is the formulation of the polyurethane polymer-modified starch:

polyurethane prepolymer, which may be added with a composition of 20 to 95% by weight, related to the total weight of the composite.

starch, which can be added in amounts ranging from 5 to 80% by weight, based on the total weight of the composite.

foaming agent, whose concentration ranges from 0.5 to 2% by weight based on the weight of polyurethane prepolymer.

Water, which is added in amounts ranging from 0.2 to 1.5% by weight, based on the polyurethane prepolymer.

In a second aspect, the preparation method of packaging material consists of mixing all the components during enough time to get a total incorporation. Afterward, the composite is left to foam until a free tack surface is obtained at a temperature range of 10 to 45° C. and then left to soak in water during 24 hours, previously to be used. It is noted that the polymerization temperature of packing material is from 10 to 50° C. being preferred temperature between 20 to 45° C. Additionally, the foaming temperature is between 10 and 50° C. Finally, the drying temperature ranges from 25 to 70° C. being preferred temperature between 40 to 60° C.

Once the polymer has been dried, it can adapt its shape by some physical means, such as but not limited to pressure or cut, according to user needs.

Polyurethane prepolymers useful for the present invention are selected from the group comprising, for example, products based on polyurethane prepolymers from polyester or polyether (Bayer Corporation, Pittsburgh, Pa., USA), polyurethane prepolymer based on polyester (NeoResins, Wilmington, Mass., USA) and polyurethane aqueous dispersions based on polyols and/or polyesters (Crompton Corporation, Greenwich, Conn., USA).

In one embodiment of the present invention, preferred are polyurethane prepolymers containing free isocyanate groups (NCO) from 10 to 18% by weight, most preferably the aliphatic polyisocyanates. An example of polyurethane prepolymer is, but is not limited to a polyisocyanate prepolymer based on diphenylmethylene isocyanate.

The starch used in the present invention can come from any plant source, being in a preferred embodiment, but not limiting the scope of the present application, corn starch, oat, potato or rice, without any other additive and especially dry.

The additives used in the present invention comprise a foaming agent and water. The foaming agent is required to obtain a foamed material that facilitates the interaction between the flow of contaminated air and the inoculated microorganisms within the material. Among foaming agents, amines, such as tertiary amines, are effective in the system described herein. The content of foaming agent in the formulation of the packing material is in the range of 0.5 to 2.0% by weight, based on the total content of the formulation. Preferred amine compounds are amine oxides which can be selected, but are not limited to the group consisting of: coconut dimethyl amine oxide; dimethyl lauryl amine oxide, decyl dimethyl amine oxide, alkyl dimethyl amine oxide, being more preferred the compounds based on lauryl amine oxide.

Regarding water as an additive. it is added for polyisocyanate prepolymer polymerization, with the aim of providing an active proton to react with the NCO groups of the polyisocyanate prepolymer.

Having described the packing material comprising a starch-modified polyurethane should be mentioned that also serves as support for microorganisms and as carbon and energy source to accelerate the formation of initial biomass. The material has sorption capacity of volatile and/or semi-volatiles organic compounds present in gaseous or liquid effluents. The packing material is inoculated with microorganisms to degrade volatile and semi-volatiles organic compounds present in gaseous or liquid effluents.

It was found that the addition of starch to polyurethane prepolymer forms a polymeric matrix that provides a source of carbon and energy for the inoculated microorganisms in such material. Such material can be used as support material of microorganisms in biological systems, for example, a biofiltration system or some other systems mentioned above. Furthermore, starch gave the polyurethane water retention capacity without it losing its hydrophobic character altogether.

Thus, the present invention provides a polymeric material capable of supporting microorganisms that can carry out biodegradation of volatile and semi-volatiles organic compounds present in gaseous or liquid effluents. The packaging material can be used in different configurations of bioreactors, for example: biofilters, drained bed biofilters, two-phase portioned solid-liquid bioreactor, fluidized bed bioreactor, among others. These bioreactors can have different geometrical shapes being these cylindrical, squares, rectangular.

Additionally, this invention provides a packaging material that can be inoculated with microorganisms, which will grow in a faster way shortening the starting time of the bioreactor during the biodegradation of volatile organic compounds due to the contents of easily assimilable carbon source as starch. So that improves the efficiency of removal of volatile and/or semi-volatile organic pollutants present gaseous or liquid effluents, and therefore, the bioreactor performance is improved.

Furthermore, The present invention provides a packaging material for the biodegradation of volatile and semi-volatile organic compounds present in gaseous or liquid effluents, which can be used in a bioreactor in continuous mode or in batch.

The packaging material of the present invention is effective to be inoculated with microorganisms and promote the growth of such microorganisms and allowing biofiltration and subsequent biodegradation of volatile organic compounds with shorter lag times, high removal efficiencies and rates of biodegradation. Furthermore, the use of this material in the biofilter showed to have high mechanical and chemical stability, which is proven to maintain high removal efficiencies for long periods of operation in the bioreactor.

Considering the above, the packaging material and method of preparation described above, conform a biofiltration system by adding microorganisms capable of biodegrading and volatile and/or semi-volatile organic compounds present in gaseous effluents or liquids.

The following describes some examples of formulations of starch-modified polyurethane packing materials as well as the results of biodegradation of compounds obtained using such formulations. It should be noted that these examples are not intended in any way limiting the scope of the invention but illustrating some of the best methods and formulations thereof.

### Example 1

### Formulation A Packing Material

100 grams of sample was prepared in a glass flask at room temperature, with the following composition: 90% by weight (w/w) polyisocyanate prepolymer with free NCO content in a range of 14.8-16.2%: 10% weight (w/w) of corn starch, 2% by weight (w/w) foaming agent (in relation to total formulation weight) 0.25% by weight (w/w) water (related to the weight of polyisocyanate). The mixing sequence was as follows: the polyisocyanate and the starch were mixed for 20 seconds, after the foaming agent was added and mixed for 40 seconds and then water was added and mixed for 1 minute. After the mixture was allowed to freely foam until its surface had no stickiness. After packing material was allowed to stand at room temperature for 24 hours, and subsequently dried at 50° C. in an oven for 1 hour.

### Example 2

### Packing Material Formulation B

100 grams of sample was prepared in a glass flask at room temperature, with the following composition: 80% by weight (w/w) polyisocyanate prepolymer with free NCO content in a range of 14.8-16.2%, 20% weight (w/w) of corn starch, 2% by weight (w/w) of foaming agent (in relation to total formulation weight) 0.25% by weight (w/w) of water (relative to the polyisocyanate weight). The mixing sequence was as follows: the polyisocyanate and the starch were mixed for 20 seconds, after the foaming agent was added and mixed for 40 seconds and then water was added and mixed for 1 minute. Subsequently, the mixture was allowed to freely foam until its surface had no stickiness. Then, the packing material was allowed to stand at room temperature for 24 hours, and subsequently dried at 50° C. in an oven for 1 hour.

### Example 3

### Formulation C Packing Material

100 grams of sample was prepared in a glass flask at room temperature, with the following composition: 70% by weight (w/w) polyisocyanate prepolymer with free NCO content in a range of 14.8-16.2%; 30% weight (w/w) of corn starch; 2% by weight (w/w) of foaming agent (in relation to total formulation weight) 0.25% by weight (w/w) of water (relative to the polyisocyanate weight). The mixing sequence was as follows: the polyisocyanate and the starch were mixed for 20 seconds, after the foaming agent was added and mixed for 40 seconds and then water was added and mixed for 1 minute. Subsequently, the mixture was allowed to freely foam until its surface had no stickiness. Then, the packing material was allowed to stand at room temperature for 24 hours, and subsequently dried at 50° C. in an oven for 1 hour.

### Example 4

### Formulation D Packaging Material

100 gram sample was prepared in a glass flask at room temperature, with the following composition: 60% by weight (w/w) polyisocyanate prepolymer with free NCO content in the range of 14.8-16.2%; 40% by weight (w/w) of corn starch; 2% by weight (w/w) of foaming agent (in relation to total formulation weight) 0.25% by weight (w/w) water (relative to the polyisocyanate weigh). The mixing sequence was as follows: the polyisocyanate and the starch were mixed for 20 seconds, after the foaming agent was added and mixed for 40 seconds and then water was added and mixed for 1 minute. Subsequently, the mixture was allowed to freely foam until its surface had no stickiness. Then, the packing material was allowed to stand at room temperature for 24 hours, and subsequently dried at 50° C. in an oven for 1 hour.

### Example 5

### Packing Material Formulation E

100 grams of sample was prepared in a glass flask at room temperature, with the following composition: 40% by weight (w/w) polyisocyanate prepolymer with free NCO content in the range of 14.8-16.2%; 60% by weight (w/w) of corn starch, 2% by weight (w/w) of foaming agent (in relation to total formulation weight) 0.25% by weight (w/w) of water (relative to the polyisocyanate weigh). The mixing sequence was as follows: the polyisocyanate and the starch were mixed for 20 seconds, after the foaming agent was added and mixed for 40 seconds and then water was added and mixed for 1 minute. Subsequently, the mixture was allowed to freely foam until its surface had no stickiness. Then, the packing material was allowed to stand at room temperature for 24 hours, and subsequently dried at 50° C. in an oven for 1 hour.

### Example 6

### Formulation F Packing Material

100 grams of sample was prepared in a glass flask at room temperature, with the following composition: 20% by weight (w/w) polyisocyanate prepolymer with free NCO content in a range of 14.8-16.2%; 80% in weight (w/w) of corn starch; 2% by weight (w/w) of blowing agent (in relation to total formulation weight) 0.25% by weight (w/w) of water (relative to the polyisocyanate weight). The mixing sequence was as follows: the polyisocyanate and the starch were mixed for 20 seconds, after the foaming agent was added and mixed for 40 seconds and then water was added and mixed for 1 minute. Subsequently, the mixture was allowed to freely foam until its surface had no stickiness. Then, the packing material was allowed to stand at room temperature for 24 hours, and subsequently dried at 50° C. in an oven for 1 hour.

### Example 7

### Packing Material Formulation G

100 grams of sample was prepared in a glass flask at room temperature, with the following composition: 100% by weight (w/w) polyisocyanate prepolymer with free NCO content in a range of 14.8-16.2%; 2% weight (w/w) of foaming agent (in relation to total formulation weight); 0.25% by weight (w/w) of water (relative to the polyisocyanate weight). The mixing sequence was as follows: the polyisocyanate and blowing agent were mixed for 20 seconds, then water was added and mixed for 1 minute. Subsequently, the mixture was allowed to freely foam until its surface had no stickiness. Then, the packing material was allowed to stand at room temperature for 24 hours and subsequently dried at 50° C. in an oven for 1 hour.

### Example 8

### Characterization of the Formulations

Since one of the most important characteristics of the packing material of the present invention is its ability to retain water, which, in turn, maintains the activity of the microorganisms capable of biodegrading volatile and semi-volatile organic compounds present in gaseous effluents or liquids, it was determined that water holding capacity (WHC) by dipping in distilled water a quantity of the material synthesized, previously weighed in dry (W_{D}). The material was allowed immersed for 48 hours. After about 48 hours, the material was removed and the wet weight of the sample was measured (W_{W}). The water holding capacity was assessed using the following expression:

% WRC=[(W_{W}−W_{D})/W_{D}]×100

The results obtained are shown in Table 1.

TABLE 1
Water holding capacity of the modified polyurethane and
polyurethane unmodified.

| Formulation | % WRC |
|---|---|
| A | 23.0 |
| B | 26.5 |
| C | 32.0 |
| D | 34.0 |
| E | 57.0 |
| F | 61.0 |
| G | 12.5 |

As seen in Table 1, addition of starch improves the water retention capacity of the polyurethane, as a consequence of the hydroxyl groups of starch, which can establish hydrogen bonding with water molecules. Water retention is an important feature because it promotes aerobic conditions and moisture in the bioreactor, necessary to sustain growth and activity of microorganisms.

Another, important aspect to consider in relation to the packing material is the mechanical resistance, since it must not occur compaction of the material over time, in order to avoid clogging and high back-pressure, which reduce biofilter performance when operated for long times.

In this, sense, the material synthesized in the present invention exhibit more elastomeric behavior as the starch content was reduced and more rigid as the starch content increased. Thus, the material with 80% of starch resulted into a brittle packing material, meanwhile the packing material with 40% of starch showed an adequate mechanical behavior, in terms of elastic recovery and resistance to stress, which has been made without compaction.

With the purpose of establishing the removal efficiency and capacity of elimination of volatile organic compounds tested with different packaging materials synthesized according to the formulations described, the following examples describe the elimination and removal efficiencies of contaminants with different Packaging materials synthesized according to Examples 1 to 7.

### Example 9

140 mg of material synthesized according to the formulation B (see Example 2), and inoculated with 50 microliters of the mixture of mineral medium and microorganisms, were placed in a glass flask with a volume of 120 milliliters. The glass flask was closed using Mininert® Teflon valves. Afterward, 4 g m⁻³ of toluene, 4 g m⁻³ of hexane and 4 g m⁻³ of methyl ethyl ketone (MEK) were added to flask, at the same time, and incubated at 28° C. The experiment was conducted by triplicate.

The concentration of toluene, hexane and MEK was monitored every day. In addition, the carbon dioxide (CO₂) concentration, into the headspace of the flasks, was measured at the beginning and at the end of the experiment, with the purpose of confirming the complete oxidation of the pollutants tested. Qualitative and quantitative analysis of biomass concentration were performed at the end of the experiment.

### Example 10

140 milligrams of material synthesized according with formulation C (see example 3), and inoculated with 50 microliters of the mixture of mineral medium and microorganisms, were placed in a glass flask with a volume of 120 milliliters. The flask was closed with Mininert® Teflon valves. Afterward, 4 g m⁻³ of toluene, 4 g m⁻³ of hexane and 4 g m⁻³ of methyl ethyl ketone (MEK) were added to flask, at the same time, and incubated at 28° C. The experiment was conducted by triplicate.

The concentration of toluene, hexane and MEK was measured daily. Furthermore, the concentration of carbon dioxide (CO2), on top of the container (void volume), was measured at baseline and end of the experiment, with the purpose of confirming the complete oxidation of the pollutants tested. We conducted both qualitative and quantitative biomass at the end of the experiment.

### Example 11

140 mg of material synthesized according to the formulation D (see Example 4), and inoculated with 50 microliters of the mixture of mineral medium and microorganisms, were placed in a glass flask with a volume of 120 milliliters. The glass flask was closed using Mininert® Teflon valves. Afterward, 4 g m⁻³ of toluene, g m⁻³ of hexane and g m⁻³ of methyl ethyl ketone (MEK) were added to flask, at the same time, and incubated at 28° C. The experiment was conducted by triplicate.

The concentration of toluene, hexane and MEK was measured daily. Furthermore, the concentration of carbon dioxide (CO2), on top of the container (void volume), was measured at start and end of the experiment, with the purpose of confirming the complete oxidation of the pollutants tested. Is qualitative and quantitative analyzes of biomass the end of the operation of biodegradation processes mentioned above.

### Example 12

140 mg of material synthesized according to the formulation G (see Example 7), and inoculated with 50 microliters of the mixture of mineral medium and microorganisms, were placed in a glass vessel with a volume of 120 ml, was added and after 50 microliters of mineral medium on the material synthesized. The flask was closed with Mininert® Teflon valves. Afterward, 4 g m⁻³ of toluene, g m⁻³ of hexane and g m⁻³ of methyl ethyl ketone (MEK) were added to flask, at the same time, and incubated at 28° C. The experiment was conducted by triplicate.

In the experiments described in Examples 9 to 12, the concentration of toluene, hexane and MEK was measured daily. Furthermore, the concentration of carbon dioxide (CO₂), on top of the container (void volume), was measured at start and end of the experiment, with the objective of corroborate the complete oxidation of the pollutants tested. Is qualitative and quantitative analyzes of biomass at the end of the operation of the biodegradation process mentioned above.

In relation to the variation of concentration with time, compared to the initial concentration (C/Co) of volatile organic compounds for Examples 9 to 12, this was followed for 15 days, and onsumption of the contaminant ie was registered.

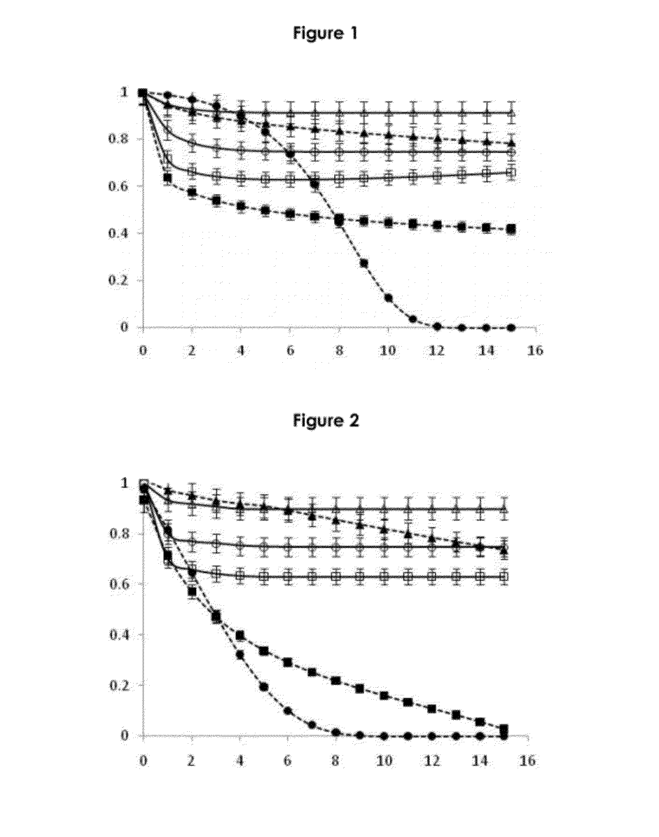

In FIG. 1, shows a reduction in the concentration of the volatile organic compounds tested in the control experiments without microorganisms, only with the packing material of Formulation G and volatile compounds, indicating that initially there was a sorption mechanism of the three compounds in the material. However, it is noted that the material was saturated with pollutants, in less than 24 hours, so that from this time, the process predominated in the biodegradation, which was corroborated by measurements of carbon dioxide at the end of experiment. Observing, the behavior of the three pollutants, in terms of their change in concentration over time, can be seen that hexane and toluene showed a rapid decline in concentration from day 1 of the experiment, unlike the MEK, which had a perceptible biodegradation after day 3, although the biodegradation of this compound was complete.

In FIG. 2, can be observed that formulation B showed higher rates of biodegradation than with formulation G, however, yielded only 27% of degradation of hexane. The toluene was completely degraded by day 15, while MEK was completely biodegraded from day 7. This increase in degradation rates can be attributed to the addition of 20% starch during polymerization of the polyurethane (formulation B). This is because the starch was used as carbon and energy source for the microorganism inoculated in the material of formulation B, and thus, promoted rapid growth of these, and consequently the increase in the rate of biodegradation of the VOC's.

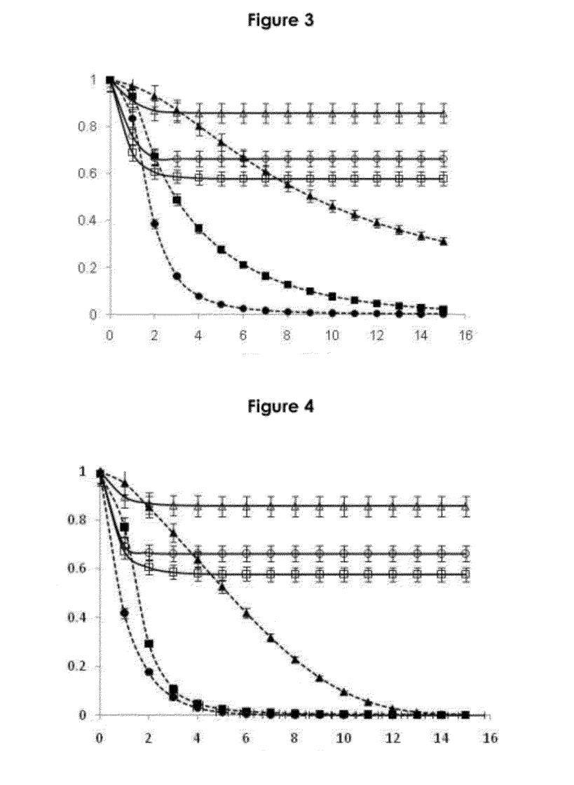

In FIG. 3, shows that with formulation C, the biodegradation of hexane was favored notoriously, as 69% of biodegradation was obtained by day 15. As for toluene, it was also completely degraded by day 15, but in a span of 9 days reached 90% biodegradation, which was faster than when using the formulation B. While the MEK, was completely degraded in 7 days, also faster than with formulation B. It is also important to note that for this composite, the rate of biodegradation of MEK changed dramatically over the formulation B, being even higher than the biodegradation of toluene.

As observable in FIG. 4, where Formulation D was used, the MEK was degraded in its entirety in only 6 days, while toluene was degraded completely in 7 days. Is important to note that only by using this formulation, complete biodegradation of hexane was achieved at day 14.

From the data shown in FIGS. 1 to 4, it can be said that the starch content in the synthesized material has a significant and noticeable effect in the biodegradation of volatile organic compounds, because it promotes the growth of biomass, which is reflected in an increased rate of biodegradation of volatile compounds, meaning that these parameters can be associated with the amount of starch present in the synthesized material based on polyurethane according to the formulations B, C and D.

Furthermore, this result prove that despite the starch is considered a easily biodegradable carbon source, its presence on the packaging material did not promote the onset of the process of catabolic repression by the use of VOCs by microorganisms, i.e., the microorganisms consumed the starch and VOCs at the same time. The above behavior is of great importance for the application of this packing material on an industrial scale, since on one hand favors the growth of biomass that has a direct effect on the reduction of the starting time of biofiltration systems and on the other side, the physicochemical properties of synthesized composite increased the sorption of VOCs implying a greater contact between the pollutant and microorganisms, which results in increased rates of biodegradation. Furthermore, due to the content of hydroxyl groups in the composite conferred by starch, microbial activity is promoted due to high water retention capacity of the composite, even the contents of these hydroxyl groups somewhat increased interactions composite with more soluble VOCs (methyl ethyl ketone and toluene), all as mentioned above in order to make a more efficient process of biodegradation of VOCs. Together with the fact that the synthesized material is capable of serving as a damper mean for load variations in the organic streams of volatile and semi-volatiles organic pollutants present in gaseous and liquid effluents, given its sorption capacity thereof.

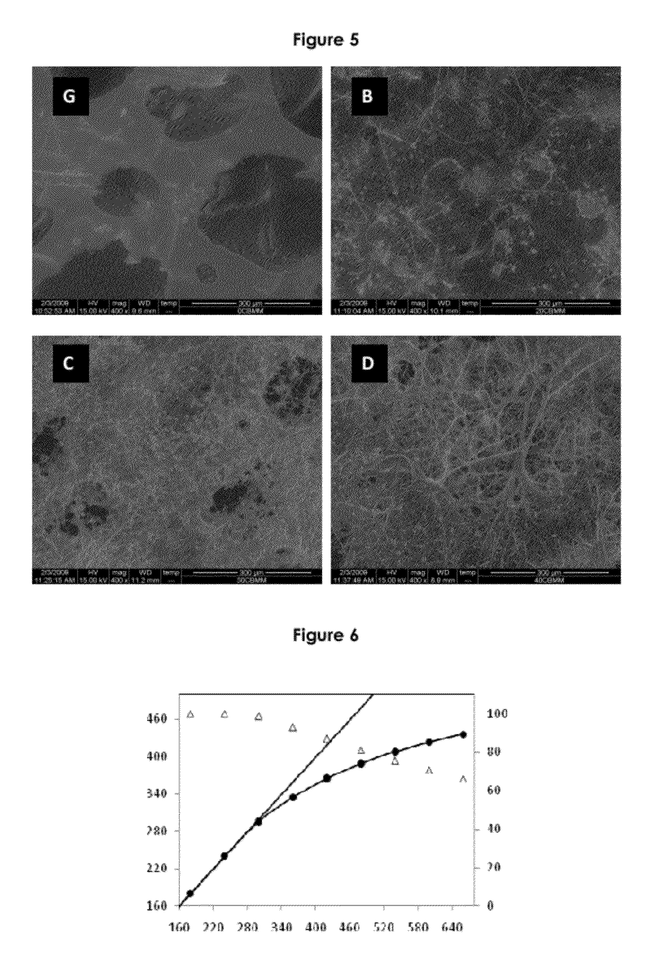

In order to corroborate the effect of starch content in the biomass growth, morphological analysis was performed by scanning electronic microscopy for samples of formulations B, C, D and G at the end of the experiment of biodegradation. The images are shown in FIG. 5.

FIG. 5 shows a significant growth of biomass related to the increase in starch content in the packaging materials. Thus, the starch promotes the biomass growing, as it acts as carbon and energy source for the microorganisms, when inoculated in the packing material. This promotes rapid formation of a stable biofilm, and therefore, as already mentioned, the starting time of the biodegradation of volatile organic compounds is substantially reduced. Further, the quantification of protein (biomass) in the materials, B, C, D and G, prove that the material D has the highest biomass content compared to, B, C and G (see Table 2).

TABLE 2
Final biomass concentration in the material of the formulations B, C,
D and G.

| | Final biomass |
|---|---|
| Formulation | (mgₚᵣₒₜₑᵢₙ/g_{dried material}) |
| G | 0.43 ± 0.0 |
| B | 1.87 ± 0.05 |
| C | 2.11 ± 0.11 |
| D | 2.76 ± 0.46 |

### Example 13

### Biofiltration System

In order to corroborate that the material of Formulation D is an excellent packing material, 20 grams of synthesized material according to the formulation D and inoculated with the mixture of mineral medium and microorganisms, were placed in a glass column (biofilter) with a volume of 100 milliliters. The biofilter had an upper inlet port and a lower output port of the flow of contaminated air stream with a mixture of volatile organic compounds, specifically hexane, toluene and methyl ethyl ketone, to a total load of 180 grams of volatile organic compounds per cubic meter of packed material in one hour. Through the top port was fed the contaminated air stream, which once passed through the packing material was dislodged by the bottom port. The biofilter was operated for 70 days in continuous mode with a continuous flow of the air stream contaminated with volatile organic compounds, with a void bed residence time of 1 min.

The concentration of VOCs was measured daily at ports of entry and exit of the flow of contaminated air. In addition, we determined the concentration of carbon dioxide (CO₂). to corroborate the complete oxidation of the pollutants. Is qualitative and quantitative analyzes of biomass at the end of the operation of biofiltration process.

The result obtained for the biofilter performance using the packing material corresponding to the formulation D is shown in FIG. 6, where can be appreciated that the maximum Elimination Capacity (EC) was 435 g m⁻³ h⁻¹ with a 73% elimination of the mixture of toluene, MEK and hexane fed. The result shows that the biofilter packed with material of Formulation D has an excellent performance in terms of efficiency of biodegradation of volatile organic compounds.

The obtained Elimination capacity values were higher than those reported by Shim, C. et al, 2006 (Environ. Sci. Technol. 2006, 40: 3089-3094), who studied different packing materials for biofiltration of an organic compounds mixture made of benzene, toluene and xylene. They reported a maximum EC of 340 g m⁻³ h⁻¹ using a biofilter packed with commercial polyurethane.

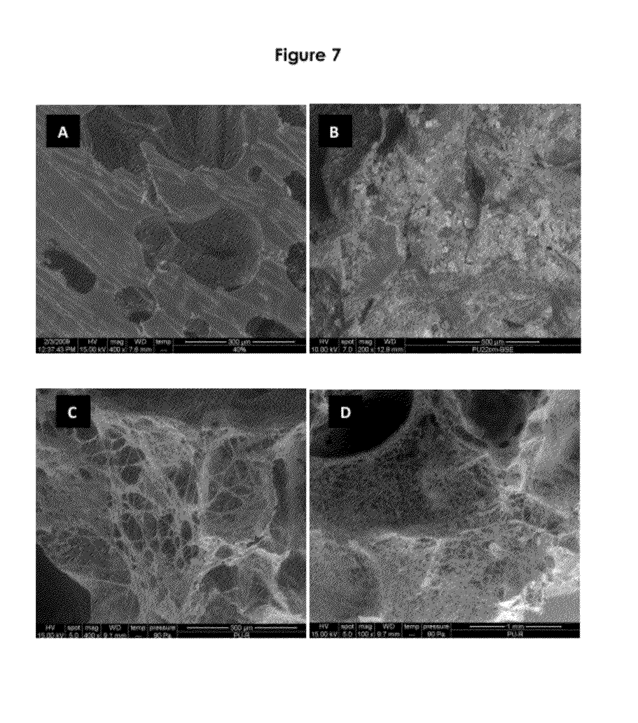

FIG. 7 shows a micrograph of the material of Formulation D, the end of the operation of the biofilter. It can be appreciated that the microorganisms biofilm is not in excess forming multilayers, i.e., there is just one layer of fungi mycelium covering the material. (monolayer).

Furthermore, in FIG. 7 can be observed that the microorganisms are essentially fungi covering the particle of the polymeric packing material. Shows the formation of intersected hyphae covering the polymeric material, as during the fed batch experiments. Another advantage of this material is that due to porosity the biomass growth during continuous operation occurred on the circumference of the pores of the material, so that there was no impediment to flow of the waste stream, ie, did not show clogging of the column, and therefore remained high efficiency of biodegradation, for long periods of operation. This corroborates that the polyurethane modified packaging system is capable of withstanding high loads without the occurrence of clogging of the column, and without presenting problems of mass transfer of contaminants and oxygen to the biofilm. In addition, compaction of the bed measured on day 70 of operation of the bioreactor was zero cm, indicating that the bed packed with the material of Formulation D, is capable of keep functioning as a carrier material for microorganisms for extended periods of time without undergoing compaction of the bed.

1. A packing material comprising a polyurethane polymer and starch, a foaming agent and water.

2. The packing material according to claim 1, wherein the polyurethane polymer is between 20% and 95% by weight of the packing material.

3. The packing material according to claim 1, wherein said polyurethane polymer is obtained from a polyurethane-based prepolymer containing 10 to 18% of free isocyanate groups.

4. The packing material according to claim 3, wherein said polyurethane-based prepolymer was selected from the group consisting of polyurethane-based prepolymers based on polyester or polyether, polyurethane prepolymer from polyesters and polyurethane aqueous dispersions from polyols and/or polyesters.

5. The packing material according to claim 1, wherein the starch is between 5% and 80% by weight.

6. The packing material according to claim 5, wherein the starch is obtained or derived from a vegetable source.

7. The packing material according to claim 6, wherein said vegetable source is corn, oats, potatoes or rice.

8. The packing material according to claim 1, comprising between 0.5% and 2.0% by weight of the foaming agent.

9. The packing material according to claim 8, wherein said foaming agent is an amine compound.

10. The packing material according to claim 9, wherein said amine compound is an amine oxide.

11. The packing material according to claim 10, wherein said amine oxide is selected from the group consisting of coconut dimethyl amine oxide, dimethyl lauryl amine oxide, decyl dimethyl amine oxide, and alkyl dimethyl amine oxide.

12. The packing material according to claim 1, comprising between 0.25% and 1.0% by weight of the water related to the polyurethane polymer weight in dry basis.

13. The packing material according to claim 1, wherein the packing material has a water retention capacity between 12% and 61% by weight of the packing material in dry basis.

14. A method for preparing a packing material, comprising the steps of:
a) mixing a polyurethane prepolymer, starch, a foaming agent, and water to form a mixture,
b) foaming the mixture, and
c) drying.

15. The method for preparing a packing material according to claim 14, wherein said polyurethane-based prepolymer contains between 10% and 18% of free isocyanate groups.

16. The method for preparing a packing material according to claim 15, wherein said polyurethane-based prepolymer is selected from the group consisting of products of polyurethane prepolymers based on polyester or polyether, polyurethane prepolymer from polyesters and polyurethane aqueous dispersions from polyols and polyesters.

17. The method for preparing a packing material according to claim 14, comprising between 5% and 80% by weight of the starch related to the packing material weight in dry basis.

18. The method for preparing a packing material according to claim 17, wherein said starch is derived from a vegetable source.

19. The method for preparing a packing material according to claim 18, wherein said vegetable source is corn, oats, potatoes or rice.

20. The method for preparing a packing material according to claim 14, wherein the foaming agent is between 0.5% and 2.0% by weight of the packing material weight in dry basis.

21. The method for preparing a packing material according to claim 20, wherein said foaming agent is an amine compound.

22. The method for preparing a packing material according to claim 21, wherein said amine compound is an amine oxide.

23. The method for preparing a packing material according to claim 22, wherein said amine oxide is selected from the group consisting of coconut dimethyl amine oxide, dimethyl lauryl amine oxide, decyl dimethyl amine oxide, and alkyl dimethyl amine oxide.

24. The method for preparing a packing material according to claim 14, wherein the packing material contains water between 0.25% and 1.0% by weight related to the packing material weight in dry basis.

25. The method for preparing a packing material according to claim 14, wherein the packing material it has a water retention capacity between 12% and 61% by weight, based on the dry weight of the packing material.

26. The method for preparing a packing material according to claim 14, wherein said mixing step uses a mechanical mixed process.

27. The method for preparing a packing material according to claim 14, wherein the mixing step is performed at a temperature between 10° C. and 50° C.

28. The method for preparing a packing material according to claim 14, wherein the foaming step is performed at a temperature between 10° C. and 50° C.

29. The method for preparing a packing material according to claim 14, wherein the drying step is performed at a temperature between 30° C. and 70° C.

30. The method for preparing a packing material according to claim 14, comprising the additional step of shaping the packing material.

31. The method for preparing a packing material according to claim 30, wherein the shape of the packing material is formed by pressure or cutting.

32. A biofiltration system comprising:
a container containing a packing material as claimed in claim 1,
a culture medium,
microorganisms capable of degrading volatile and semi-volatile organic compounds present in gaseous or liquid effluents, and
the container has inlet and outlet ports to admit and release compounds.

33. The biofiltration system according to claim 32, wherein said culture medium is a mineral salts medium.

34. The biofiltration system according to claim 32, wherein the microorganism are bacteria, yeasts or fungi or mixture thereof.

35. The biofiltration system according to claim 32, wherein the system is fed with volatile organic compounds, individual or mixed, with organic loading rates ranging from 1 g m⁻³ h⁻¹ to 640 g m⁻³ h⁻¹.

36. The biofiltration system according to claim 32, wherein the biodegradation efficiency of volatile and semi-volatile organic compounds is at least 85% compared to the initial concentration of such compounds.
